(19) United States
(12) Patent Application Publication
(10) Pub. No.: US 2017/0245834 A1
(43) Pub. Date: Aug. 31, 2017

(54) Ultrasonic Diagnostic Apparatus, Method Of Controlling Ultrasonic Diagnostic Apparatus, And Program

(71) Applicant: KONICA MINOLTA, INC., Chiyoda-ku (JP)

(72) Inventor: Yoshihiro TAKEDA, Hachioji-shi (JP)

(73) Assignee: KONICA MINOLTA, INC., Chiyoda-ku (JP)

(21) Appl. No.: 15/438,206

(22) Filed: Feb. 21, 2017

(30) Foreign Application Priority Data
Feb. 26, 2016 (JP) 2016-035283

(51) Int. Cl. A61B 8/08; A61B 8/00

(52) U.S. Cl. CPC A61B 8/5223; A61B 8/4444; A61B 8/461; A61B 8/485; A61B 8/54

(57) ABSTRACT

An ultrasonic diagnostic apparatus includes: an ultrasonic probe which transmits an ultrasonic wave to a subject and receives a reflected wave of the transmitted ultrasonic wave; a transmission/reception control unit which allows the ultrasonic probe to transmit the ultrasonic wave and acquires a reception signal relating to the ultrasonic wave received by the ultrasonic probe on the basis of predetermined transmission/reception setting; a calculation parameter acquisition unit which acquires a calculation parameter which is used for generation of elasticity information in the subject based on the reception signal and is determined in correspondence with the transmission/reception setting; an elasticity information generation unit which generates the elasticity information by using the reception signal on the basis of the acquired calculation parameter; and a display control unit which allows the display unit to display the elasticity information.

The entire disclosure of Japanese Patent Application No. 2016-035283 filed on Feb. 26, 2016 including description, claims, drawings, and abstract are incorporated herein by reference in its entirety.

### BACKGROUND OF THE INVENTION

Field of the Invention

The present invention relates to an ultrasonic diagnostic apparatus, a method of controlling an ultrasonic diagnostic apparatus, and a program.

Description of the Related Art

In the related art, there is an ultrasonic diagnostic apparatus which provides diagnostic information of an internal structure of a subject by irradiating an inner portion of the subject with an ultrasonic wave from an ultrasonic probe, receiving a reflected wave, processing the obtained reception signal to generate an ultrasonic image reflecting the internal structure of the subject, and displaying the ultrasonic image on a display unit. Such an ultrasonic diagnostic apparatus is also used for interpersonal medical care as a noninvasive diagnostic apparatus.

With respect to an ultrasonic diagnostic apparatus, there is a technique of generating elasticity information reflecting hardness of an internal structure of a subject and displaying the elasticity information on the display unit. The elasticity information is displayed, for example, as an elastic image colored according to hardness of each portion of the subject. The elasticity information is generated by transmitting an ultrasonic wave while pressurizing the subject and calculating distortion of the subject from reception signals of reflected waves at two time points in different pressurized states. For generation of the elasticity information, there may be used, for example, a method of calculating the distortion by scanning and transmitting the ultrasonic wave at a predetermined frame frequency while pressurizing the subject with a time-varying pressure and comparing and analyzing reception signals of reflected waves in two frames at different times.

In some cases, since distortion of the subject in two frames for generating the elasticity information is too small or too large according to a time rate of change of the pressure of pressurizing the subject and a size of the frame frequency, the distortion cannot be properly calculated, and appropriate elasticity information cannot be obtained. On the other hand, JP 2004-261198A discloses a technique for selecting a frame to be used for calculating distortion from frames other than consecutive frames. In addition, JP 2009-148593 A discloses a technique for changing a frame frequency so as to appropriately calculate distortion.

However, if the interval of frames used for generating the elasticity information is changed, in many cases, the quality of elasticity information such as distortion calculation accuracy and resolution is deteriorated. On the other hand, in order to improve the deterioration of the quality of the elasticity information, it is necessary to adjust a calculation parameter used for the setting relating to the transmission and reception of an ultrasonic wave and the analyzing of the reception signal every time, and thus, it takes time and effort.

As described above, in the related art, there is a problem that appropriate elasticity information cannot be easily generated.

### SUMMARY OF THE INVENTION

An object of the present invention is to provide an ultrasonic diagnostic apparatus capable of easily generating appropriate elasticity information, a method of controlling the ultrasonic diagnostic apparatus, and a program.

To achieve the abovementioned object, according to an aspect, an ultrasonic diagnostic apparatus reflecting one aspect of the present invention comprises:

an ultrasonic probe which transmits an ultrasonic wave to a subject and receives a reflected wave of the transmitted ultrasonic wave;

a transmission/reception control unit which allows the ultrasonic probe to transmit the ultrasonic wave and acquires a reception signal relating to the ultrasonic wave received by the ultrasonic probe on the basis of predetermined transmission/reception setting;

a calculation parameter acquisition unit which acquires a calculation parameter which is used for generation of elasticity information in the subject based on the reception signal and is determined in correspondence with the transmission/reception setting;

an elasticity information generation unit which generates the elasticity information by using the reception signal on the basis of the acquired calculation parameter; and

a display control unit which allows the display unit to display the elasticity information.

According to an invention of Item. 2, in the ultrasonic diagnostic apparatus of Item. 1, the ultrasonic diagnostic apparatus preferably further comprises a storage unit which stores a plurality of the calculation parameters determined in advance in association with a plurality of different transmission/reception settings, and

the calculation parameter acquisition unit preferably acquires the calculation parameter from the storage unit.

According to an invention of Item. 3, in the ultrasonic diagnostic apparatus of Item. 1 or 2,

the elasticity information generation unit preferably generates the elasticity information by calculating distortion of the subject for each correlation calculation region corresponding to a predetermined time range between a first reception signal relating to the ultrasonic wave reflected by the subject in a first pressurized state and a second reception signal relating to the ultrasonic wave reflected by the subject in a second pressurized state by using the first reception signal and the second reception signal, and

the calculation parameter acquisition unit preferably acquires the calculation parameter indicating a size of the correlation calculation region.

According to an invention of Item. 4, in the ultrasonic diagnostic apparatus of Item. 3, the elasticity information generation unit preferably extracts a phase difference component at each time in a time range corresponding to the correlation calculation region between the first reception signal and the second reception signal and calculates the distortion of the subject in the correlation calculation region from the phase difference component and a center frequency of the ultrasonic wave transmitted from the ultrasonic probe.

According to an invention of Item. 5, in the ultrasonic diagnostic apparatus of Item. 1 or 2,

the transmission/reception control unit preferably allows the ultrasonic probe to transmit an ultrasonic wave while scanning the ultrasonic wave in a predetermined scan direction and acquires two-dimensional data of the reception signal relating to the scanned ultrasonic wave every scanning,

the elasticity information generation unit preferably generates the elasticity information by detecting a shift at each position of the subject between a first pressurized state and a second pressurized state by using first two-dimensional data relating to the ultrasonic wave reflected by the subject in the first pressurized state and second two-dimensional data relating to the ultrasonic wave reflected by the subject in the second pressurized state,

the detection of the shift is preferably performed by specifying the shift in a predetermined search region including the correlation calculation region for each of two-dimensional predetermined correlation calculation regions in the two-dimensional data, and

the calculation parameter acquisition unit preferably acquires the calculation parameter indicating at least one of a size of the correlation calculation region and a size of the search region.

According to an invention of Item. 6, in the ultrasonic diagnostic apparatus of any one of Items. 1 to 4,

the transmission/reception setting preferably includes setting of a center frequency of an ultrasonic wave transmitted from the ultrasonic probe.

According to an invention of Item. 7, in the ultrasonic diagnostic apparatus of any one of Items. 1 to 6,

the transmission/reception control unit preferably acquires the reception signal relating to the reflected wave of which reflection position on the subject is equal to or less than a predetermined maximum depth among the ultrasonic waves transmitted to the subject, and

the transmission/reception setting preferably includes setting of the maximum depth.

According to an invention of Item. 8, in the ultrasonic diagnostic apparatus of Item. 7,

the transmission/reception control unit preferably acquires the reception signal at a sampling frequency that is lower as the set maximum depth is larger.

According to an invention of Item. 9, in the ultrasonic diagnostic apparatus of any one of Items. 1 to 8,

the transmission/reception control unit preferably allows the ultrasonic probe to transmit while scanning the ultrasonic wave in a predetermined scan direction, acquires two-dimensional data of the reception signal relating to the scanned ultrasonic wave every scanning, and

the transmission/reception setting preferably includes setting of a frame frequency indicating a frequency of the scanning.

According to an invention of Item. 10, in the ultrasonic diagnostic apparatus of any one of Items. 1 to 9, the ultrasonic diagnostic apparatus preferably further comprises:

an input unit which receives an input operation for determining the transmission/reception setting; and

a transmission/reception setting change unit which determines the transmission/reception setting on the basis of the input operation.

According to an invention of Item. 11, in the ultrasonic diagnostic apparatus of any one of Items. 1 to 10,

the elasticity information is preferably an elastic image representing a distribution of values relating to distortion in the subject.

According to an invention of Item. 12, in the ultrasonic diagnostic apparatus of Item. 11, the ultrasonic diagnostic apparatus preferably further comprises an ultrasonic image generation unit which generates an ultrasonic image representing an internal structure of the subject by using the reception signal, and

the display control unit preferably allows the display unit to display the ultrasonic image and the elastic image.

According to an invention of Item. 13, in the ultrasonic diagnostic apparatus of Item. 12,

the display control unit preferably allows the display unit to display the ultrasonic image and the elastic image in a superimposed manner.

To achieve the abovementioned object, according to an aspect, a method of controlling an ultrasonic diagnostic apparatus including an ultrasonic probe which transmits an ultrasonic wave to a subject and receives a reflected wave of the transmitted ultrasonic wave, reflecting one aspect of the present invention comprises:

allowing the ultrasonic probe to transmit an ultrasonic wave and acquiring a reception signal relating to the ultrasonic wave received by the ultrasonic probe on the basis of predetermined transmission/reception setting;

acquiring a calculation parameter which is used for generation of elasticity information in the subject based on the reception signal and is determined in correspondence with the transmission/reception setting;

generating the elasticity information by using the reception signal on the basis of the acquired calculation parameter; and

allowing a display unit to display the elasticity information.

To achieve the abovementioned object, according to an aspect, there is provided a non-transitory recording medium storing a computer readable program, and the program reflecting one aspect of the present invention causes a computer to function as:

a transmission/reception control unit which allows an ultrasonic probe to transmit an ultrasonic wave to a subject and acquires a reception signal relating to a reflected wave of the ultrasonic wave received by the ultrasonic probe on the basis of predetermined transmission/reception setting;

a calculation parameter acquisition unit which acquires a calculation parameter which is used for generation of elasticity information in the subject based on the reception signal and is determined in correspondence with the transmission/reception setting;

an elasticity information generation unit which generates the elasticity information by using the reception signal on the basis of the acquired calculation parameter; and

a display control unit which allows the display unit to display the elasticity information.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is an overall diagram illustrating an ultrasonic diagnostic apparatus according to an embodiment of the present invention;

FIG. 2 is a block diagram illustrating an internal configuration of an ultrasonic diagnostic apparatus;

FIGS. 3A and 3B are diagrams for explaining distortion measurement;

FIG. 4 is a diagram for explaining a flow of distortion calculation and image generation;

FIG. 5 is a diagram for explaining a reception signal used for distortion calculation process;

FIGS. 6A to 6C are diagrams illustrating a method of setting a size of a correlation calculation region according to transmission/reception setting;

FIGS. 7A and 7B are diagrams illustrating a difference in phase difference according to a center frequency in the case where the same distortion is given;

FIG. 8 is a diagram illustrating an example of setting of a correlation calculation region according to a center frequency;

FIG. 9 is a flowchart illustrating a control procedure in an elastic image display process;

FIG. 10 is a flowchart illustrating a control procedure in an elastic image generation process;

FIG. 11 is a diagram for explaining a distortion calculation method according to a second embodiment; and

FIGS. 12A and 12B are diagrams illustrating methods of setting a size of a correlation calculation region according to transmission/reception setting in the second embodiment.

### DESCRIPTION OF THE PREFERRED EMBODIMENTS

Hereinafter, a first embodiment of the present invention will be described with reference to the drawings. However, the scope of the invention is not limited to the illustrated examples.

### First Embodiment

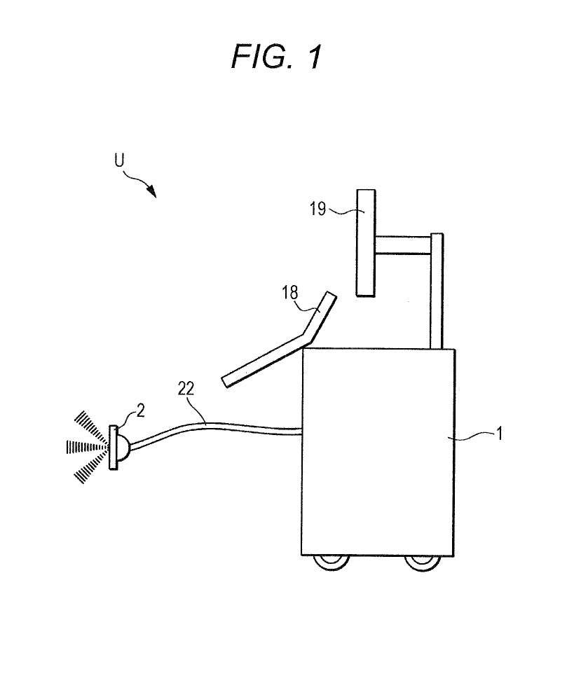

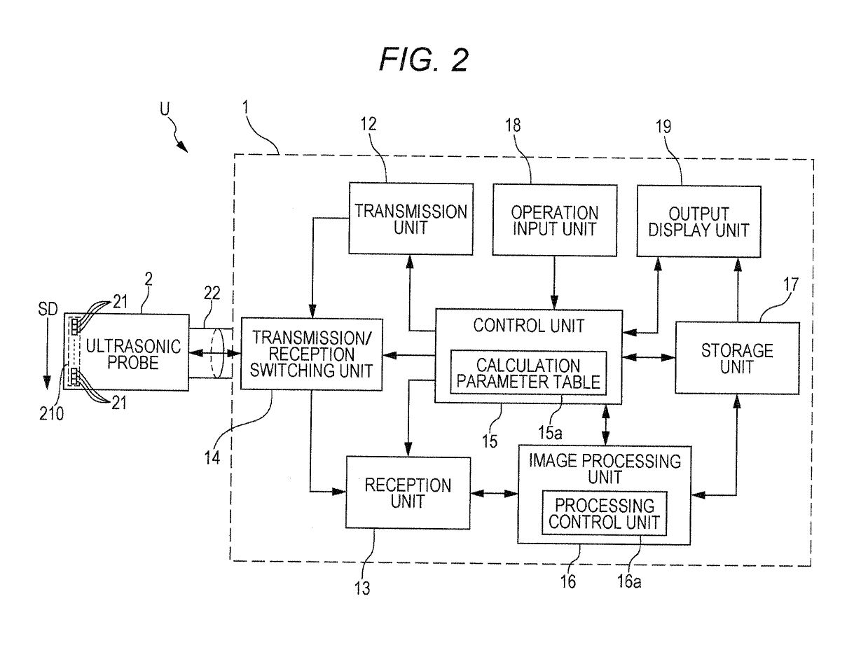

FIG. 1 is an overall diagram of an ultrasonic diagnostic apparatus U according to the embodiment. FIG. 2 is a block diagram illustrating an internal configuration of the ultrasonic diagnostic apparatus U.

As illustrated in FIG. 1, the ultrasonic diagnostic apparatus U is configured to include an ultrasonic diagnostic apparatus main body 1, an ultrasonic probe 2 connected to the ultrasonic diagnostic apparatus main body 1 through a cable 22. The ultrasonic diagnostic apparatus main body 1 is provided with an operation input unit 18 (input unit) and an output display unit 19 (display unit). The control unit 15 of the ultrasonic diagnostic apparatus main body 1 outputs a drive signal to the ultrasonic probe 2 to output an ultrasonic wave on the basis of an external input operation to an input device such as a keyboard and a mouse of the operation input unit 18. The control unit acquires a reception signal relating to reception of the ultrasonic wave from the ultrasonic probe 2 to perform various processes and displays results or the like of the processes on a liquid crystal screen or the like of the output display unit 19 as necessary. The output display unit 19 may not be included in the ultrasonic diagnostic apparatus U, but the output display unit may be provided outside the ultrasonic diagnostic apparatus U.

As illustrated in FIG. 2, the ultrasonic diagnostic apparatus main body 1 is configured to include a transmission unit 12, a reception unit 13, a transmission/reception switching unit 14, a control unit 15 (a display control unit, a transmission/reception setting change unit, and a storage unit), an image processing unit 16, a storage unit 17, an operation input unit 18, an output display unit 19, and the like. Among these components, the transmission unit 12, the reception unit 13, and the control unit 15 constitute a transmission/reception control unit.

The transmission unit 12 outputs a pulse signal to be supplied to the ultrasonic probe 2 according to a control signal input from the control unit 15 and allows the ultrasonic probe 2 to generate an ultrasonic wave having a center frequency corresponding to predetermined transmission/reception setting. The transmission unit 12 is configured to include, for example, a clock generation circuit, a pulse generation circuit, a pulse width setting unit, and a delay circuit. The clock generation circuit is a circuit for generating a clock signal for determining transmission timing of the pulse signal and the center frequency. The pulse generation circuit is a circuit for generating a bipolar rectangular wave pulse having a voltage amplitude determined in advance at a predetermined period. The pulse width setting unit sets a pulse width of the rectangular wave pulse output from the pulse generation circuit. The rectangular wave pulse generated by the pulse generation circuit is separated into different wiring paths for the respective transducers 21 of the ultrasonic probe 2 before or after input to the pulse width setting unit. The delay circuit is a circuit for delaying the generated rectangular wave pulses by respective delay times set for the respective wiring paths corresponding to timing of transmitting the generated rectangular wave pulses to the respective transducers 21 and outputting the delayed rectangular wave pulses.

The reception unit 13 is a circuit for acquiring the reception signal input from the ultrasonic probe 2 under the control of the control unit 15. The reception unit 13 includes, for example, an amplifier, an A/D conversion circuit, and a phasing addition circuit. The amplifier is a circuit for amplifying the reception signal corresponding to the ultrasonic wave received by each transducer 21 of the ultrasonic probe 2 with a predetermined amplification factor which is determined in advance. The A/D conversion circuit is a circuit for converting the amplified reception signal into digital data at a sampling frequency corresponding to predetermined transmission/reception setting. Since the Nyquist frequency needs to be higher than a reception frequency described later, for example, the sampling frequency is 60 MHz. The phasing addition circuit is a circuit for adjusting a time phase by applying a delay time for each wiring path corresponding to each transducer 21 to the A/D-converted reception signal and performing addition (phasing addition) on results thereof to generate sound line data.

In the embodiment, the sampling frequency is set so that the number of times of sampling is allowed to be constant irrespective of a depth of inspection of the subject (the maximum depth of the reflection position of the ultrasonic wave where the reception signal is acquired), that is, a length of the period for acquiring the reception signal of the ultrasonic wave. Therefore, in the case where the depth of inspection of the subject is larger, the sampling frequency is lowered according to the depth, and similarly, in the case where the depth is small, the sampling frequency is heightened. In the embodiment, the depth of inspection of the subject is set as the transmission/reception setting, and the reception unit 13 acquires the reception signal at the sampling frequency corresponding to the set depth.

Under the control of the control unit 15, in the case where the transducer 21 is to emit the ultrasonic wave, the transmission/reception switching unit 14 allows the transmission unit 12 to transmit the drive signal to the transducer 21, and in the case where the signal relating to the ultrasonic wave emitted by the transducer 21 is to be acquired, the transmission/reception switching unit performs a switching operation for allowing the reception unit 13 to output the reception signal.

The control unit 15 is configured to include a CPU (Central Processing Unit), an HDD (Hard Disk Drive), a RAM (Random Access Memory), and the like. The CPU reads out various programs stored in the HDD and loads the programs onto the RAM. The CPU centrally controls the operations of the components of the ultrasonic diagnostic apparatus U according to the programs. The HDD stores a control program for operating the ultrasonic diagnostic apparatus U, various processing programs, various set data, and the like. The programs and the set data may be stored in an auxiliary storage device using a nonvolatile memory such as a flash memory other than the HDD so as to be readable, writable, and updatable. The RAM is a volatile memory such as an SRAM or a DRAM. The RAM provides a memory space for work to the CPU and temporarily stores data.

The set data stored in the HDD of the control unit 15 includes transmission/reception setting relating to transmission and reception of an ultrasonic wave and a calculation parameter table 15a. Among the set data, the transmission/reception setting includes the center frequency of the transmission ultrasonic wave, the depth of inspection of the subject, and the frame frequency of the transmission ultrasonic wave. Therefore, the set value can be changed according to user's performing a predetermined input operation to the operation input unit 18. The calculation parameter table 15a is table data where predetermined calculation parameters are relating to respective ones of a plurality of transmission/reception settings that can be set in the ultrasonic diagnostic apparatus U. Herein, the calculation parameter is a parameter used for distortion calculation described later, and the details thereof will be described later.

Separately from the CPU of the control unit 15, the image processing unit 16 is provided with a processing control unit 16a (elasticity information generation unit and ultrasonic image generation unit) including a CPU, a RAM, and the like for performing a calculation process for generating a diagnostic image based on the reception data of the ultrasonic wave. The diagnostic image includes a B mode image (ultrasonic image) illustrating a structure of a subject according to luminance distribution, a D mode image representing a blood flow state and the like measured by using the Doppler effect, and elastic image (elasticity information) illustrating a distribution of distortion inside the subject, and the like. In addition, the diagnostic image further includes image data to be displayed on the output display unit 19 in substantially real time, a series of moving picture data thereof, still image data of snapshots, and the like.

In addition, the calculation process of the processing control unit 16a may be configured to be performed by the CPU of the control unit 15.

The storage unit 17 is, for example, a volatile memory such as a DRAM (Dynamic Random Access Memory). Alternatively, the storage unit 17 may be various nonvolatile memories that are rewritable at a high speed. The storage unit 17 stores diagnostic image data which are processed by the image processing unit 16 and are used for real-time display or display conformable thereto in units of a frame. The diagnostic image data stored in the storage unit 17 are read under the control of the control unit 15 and are transmitted to the output display unit 19 or output to the outside of the ultrasonic diagnostic apparatus U through a communication unit. At this time, in the case where the display system of the output display unit 19 is a television system, a DSC (Digital Signal Converter) may be provided between the storage unit 17 and the output display unit 19, and after the scanning format is converted, the output may be performed.

The operation input unit 18 is configured to include a push button switch, a keyboard, a mouse, a trackball, or a combination thereof and convert a user's input operation into an operation signal and input the operation signal to the ultrasonic diagnostic apparatus main body 1.

The output display unit 19 is configured to include a display screen using any one of various display systems such as an LCD (Liquid Crystal Display), an organic EL (Electro-Luminescent) display, an inorganic EL display, a plasma display, and a CRT (Cathode Ray Tube) and a driving unit thereof. The output display unit 19 generates a drive signal of the display screen (each display pixel) according to the control signal output from the CPU 15 and the diagnostic image data generated by the image processing unit 16 and displays menus or status relating to the ultrasonic diagnosis and measurement data based on the received ultrasonic wave on the display screen.

The operation input unit 18 and the output display unit 19 may be provided integrally with a housing of the ultrasonic diagnostic apparatus main body 1 or may be externally attached through a USB cable or the like. In addition, if the ultrasonic diagnostic apparatus main body 1 is provided with an operation input terminal and a display output terminal, the ultrasonic diagnostic apparatus main body 1 may be used with the configuration where operation and display peripheral devices of the related art are connected to the terminals.

The ultrasonic probe 2 oscillates an ultrasonic wave (herein, about 1 to 30 MHz) to emit (transmit) the ultrasonic wave to a subject such as a living body, and the ultrasonic probe 2 functions as an acoustic sensor that receives a reflected wave (echo) reflected on the subject among the emitted ultrasonic wave and converts the reflected wave into an electric signal. The ultrasonic probe 2 is configured to include a transducer array 210 which is an array of a plurality of transducers 21 transmitting and receiving an ultrasonic wave and a cable 22. The cable 22 has a connector (not shown) to the ultrasonic diagnostic apparatus main body 1 at one end thereof, and the ultrasonic probe 2 is configured to be detachable with respect to the ultrasonic diagnostic apparatus main body 1 through the cable 22.

The user allows the ultrasonic diagnostic apparatus U to operate and perform ultrasonic diagnosis by allowing an ultrasonic wave transmission/reception surface of the ultrasonic probe 2, that is, a surface in the direction of emitting an ultrasonic wave from the transducer array 210 to be in contact with the subject at a predetermined pressure.

The transducer array 210 is an array of the transducers 21 having a piezoelectric element having a piezoelectric body and electrodes provided at both ends where charges appear according to deformation (expansion and contraction) of the piezoelectric body. In the embodiment, the transducer array is a one-dimensional array in a predetermined scan direction SD. Voltage pulses (pulse signals) are sequentially supplied to the vibrator 21, so that each piezoelectric body is deformed according to an electric field generated in the piezoelectric body and an ultrasonic wave is transmitted. In addition, if an ultrasonic wave in a predetermined frequency band is incident on the vibrator 21, the thickness of the piezoelectric body is fluctuated (vibrated) according to a sound pressure, so that electric charges corresponding to an amount of fluctuation are generated to be converted into an electric signal, and the electric signal is outputted.

The ultrasonic probe 2 performs scanning of the ultrasonic wave in the scan direction SD parallel to the transducer array direction by transmitting the ultrasonic wave from the transducers 21 in the order of arrangement in the transducer array 210 on the basis of the pulse signal from the transmission unit 12. In the embodiment, the scanning of the ultrasonic wave is repeatedly performed at a frame frequency corresponding to the transmission/reception setting described above. In addition, in each scanning, the reception unit 13 acquires two-dimensional data (hereinafter, also referred to as frame data) of the reception signal from the reception signal (acoustic line) relating to the reflected wave received by each transducer 21.

The ultrasonic probe 2 may employ any method of various electronic scanning methods such as a linear electronic scanning method, a sector electronic scanning method, and a convex electronic scanning method and various mechanical scanning methods such as a linear scanning method, a sector scanning method, an arc scanning method, and a radial scanning method. In addition, the ultrasonic diagnostic apparatus U may be configured so that any one of a plurality of different ultrasonic probes 2 corresponding to diagnostic objects is used to be connected to the ultrasonic diagnostic apparatus main body 1. In addition, the ultrasonic probe 2 may be configured to include a pressure sensor to measure the pressure of the ultrasonic probe 2 on the subject and output the pressure to the control unit 15. The ultrasonic probe 2 may be configured to further include a motor for moving the transmission/reception surface of the ultrasonic probe 2 forward and backward in the transmission/reception direction of the ultrasonic wave, so that the ultrasonic probe 2 can press the subject with a pressure which is set in advance and the ultrasonic probe 2 can be released from the subject.

Next, a distortion measurement operation of the ultrasonic diagnostic apparatus U according to the embodiment will be described. The ultrasonic diagnostic apparatus U according to the embodiment has a B mode where one-dimensional to two-dimensional display relating to tomographic inspection is performed in a substantially real time by using luminance, a D mode where a blood flow state or the like is measured and displayed by using the Doppler effect, and an elasticity information display mode where an elastic image representing a distribution of distortion of an internal structure is displayed so as to be superimposed on the B mode image. The ultrasonic diagnostic apparatus U performs the distortion measurement operation on the subject in the elasticity information display mode among these modes.

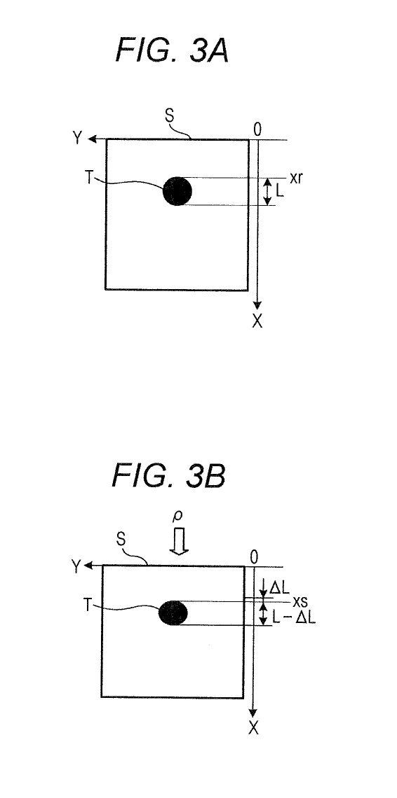

FIGS. 3A and 3B are diagrams for explaining measurement of distortion.

As illustrated in FIG. 3A, inside the subject S in the normal state, there is an upper end of a structure T at a position of a distance xr from a contact surface with an ultrasonic transmission surface of the ultrasonic probe 2 on the upper surface of the subject S in the depth direction (X direction). In addition, the width of this structure T in the X direction is L. As illustrated in FIG. 3B, if it is assumed that, in a state where a pressure ρ (stress) is applied to the subject S from the upper surface side, the same pressure p is also applied to the structure T, the upper end position of the structure T is a distance xs in the X direction, and the width is changed to L−ΔL.

Therefore, by measuring the structure T in the two states, the distortion ε=ΔL/L can be obtained. At this time, the longitudinal modulus of elasticity (Young's modulus) E=ρ/ε can be calculated by using the pressure ρ (stress) measured by a pressure sensor, and the longitudinal modulus of elasticity can also be displayed.

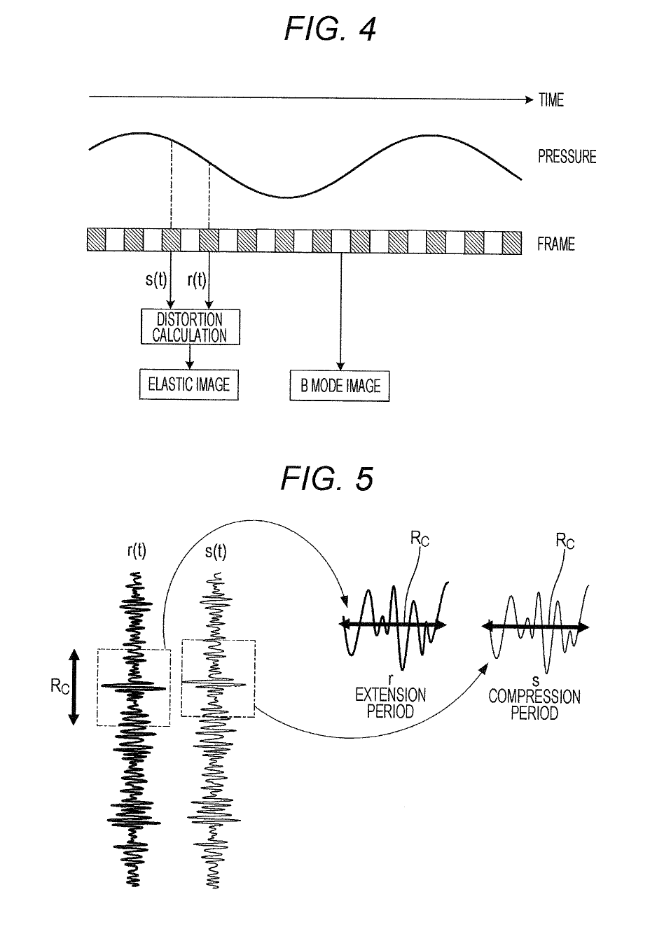

FIG. 4 is a diagram for explaining a flow of distortion calculation and image generation.

In the elasticity information display mode, an ultrasonic wave is transmitted and received while applying time-varying pressure to the subject. Change in pressure may be performed by the hand of the operator or may be realized by the pressing mechanism provided with the pressing mechanism on the ultrasonic probe 2. In addition, in the case where the subject is a living body, a mode where the pressure is changed by movement according to respiration or the like of the subject with respect to the fixed ultrasonic probe 2 may be employed. The ultrasonic wave is repeatedly scanned at a predetermined frame frequency, and frame data is acquired for each frame. Herein, in the elasticity information display mode according to the embodiment, the elastic image is generated by calculating the distortion by using frame data of an odd-numbered frame, that is, a (2n-1)-th frame (n is a natural number), and the B mode image is generated by using frame data of an even-numbered frame, that is, a 2n-th frame.

In the two adjacent frames (the above-described two frames which are different in n by 1) among the odd-numbered frame group, the subject is in a state where different pressures are applied to the same portion (a first pressurized state and a second pressurized state). Therefore, in the embodiment, the elastic image is generated by using the frame data of the two frames. Namely, the frame data of each frame excluding the first and last frames among the odd-numbered frame group are used twice for calculating the distortion. After that, the elastic image is obtained by performing a process such as smoothing or dynamic range adjusting for display on the calculated two-dimensional data of the distortion distribution. For example, the elastic image is displayed on the output display unit 19 in a color display or a gray scale display so as to be superimposed on the B mode image.

Next, the distortion calculation process in the ultrasonic diagnostic apparatus U according to the embodiment will be described.

FIG. 5 is a diagram for explaining the reception signal used for distortion calculation process.

In the ultrasonic diagnostic apparatus U according to the embodiment, among the two frames used for calculating the distortion, the reception signal of the ultrasonic wave in the frame where the pressure applied to the subject is relatively small is expressed as a waveform r(t) (first reception signal), and the reception signal of the ultrasonic wave in the frame where the pressure is relatively large is acquired as a compression-period waveform s(t) (second reception signal). On the left side of FIG. 5, examples of the extension-period waveform r(t) and the compression-period waveform s(t) are illustrated with the time axis as the vertical direction.

In the embodiment, the distortion is calculated for each waveform in a time range (hereinafter, referred to as a correlation calculation region R_{C}) corresponding to the same region in the depth direction of the subject among the extension-period waveform r(t) and the compression-period waveform s(t). Namely, as illustrated on the right side of FIG. 5, portions corresponding to one correlation calculation region R_{C} among the extension-period waveform r(t) and the compression-period waveform s(t) are extracted, respectively, and the distortion is calculated on the basis of the extracted waveforms. After that, the distortion calculation process is repeatedly performed in units of a correlation calculation region R_{C}.

Hereinafter, a distortion calculation method performed for each correlation calculation region R_{C} will be described.

The extension-period waveform r(t) at each data acquisition timing (elapsed time t (time)) in the correlation calculation region R_{C} is as follows:

r(t)=A(t)cos(ω₀t+φ(t)) (1)

wherein, ω₀ is a center frequency of the received ultrasonic wave, A (t) is a time change of the amplitude component (envelope of the received waveform), and φ(t) is an initial phase.

The waveform can be analytically expressed by a complex function as follows:

rₐ₍t)=A(t)exp(iω₀t+φ(t)) (2)

On the other hand, in the compression-period waveform s(t), the reflected wave with respect to a predetermined structure is observed in a shorter time, that is, in a shorter period than the extension-period waveform r(t) according to the distortion ε (that is, the extension ratio, ε<0 in the compression period). In addition, as pressure is indirectly applied to the subject, the position of the inner portion of the subject moves from xr to xs, so that the detection timing of the reflected wave, that is, the phase is changed. In the case where the distortion ε is within a minute range (usually, for example, 5% or less), the compression-period waveform s(t) is expressed as a waveform obtained by compressing the extension-period waveform rₐ(t) by an amount corresponding to the distortion ε, as illustrated in the following Mathematical Formula (3).

sₐ(t)=A(t(1−ε))exp(iω₀t(1−ε)+φ(t(1−ε) (3)

A phase difference Fₐ(t) (phase difference component) between the extension-period waveform rₐ(t) and the compression-period waveform sₐ(t) is obtained from the analytical solutions (2) and (3) by the following Mathematical Formula (4):

Fₐ(t)=Im(log(rₐ(t)sₐ*(t)))=δω₀t+ε (4)

wherein, sₐ*(t) is a complex conjugate of the compression-period waveform sₐ(t), and δ represents a phase shift (initial phase difference) according to the above-described difference between the distance xs and the distance xr. Namely, the phase difference Fₐ(t) is a linear function where the slope is proportional to the distortion ε and the center frequency ω₀ and the intercept is expressed by the phase shift δ.

Therefore, the distortion ε in the correlation calculation region R_{C} can be obtained from the phase difference Fₐ(t) at each time obtained from the real and imaginary parts of the measured extension-period waveform r(t) and the real and imaginary parts of the compression-period waveform s(t) and the center frequency ω₀ of the ultrasonic wave.

By performing calculation of the above-mentioned distortion ε on a plurality of the correlation calculation regions R_{C} in the depth direction of the subject, respectively, and by performing this process for each position in the scan direction of the ultrasonic wave, two-dimensional data of the distortion of the subject are acquired.

Next, the setting of the size of the correlation calculation region R_{C} according to the transmission/reception setting in the ultrasonic diagnostic apparatus U according to the embodiment will be described.

As described above, in the embodiment, the center frequency of the transmission ultrasonic wave, the depth of inspection of the subject, and the frame frequency of the transmission ultrasonic wave are set as the transmission/reception setting of the ultrasonic wave. In the calculation of the distortion, the size (calculation parameter) of an appropriate correlation calculation region R_{C} is set according to the transmission/reception setting.

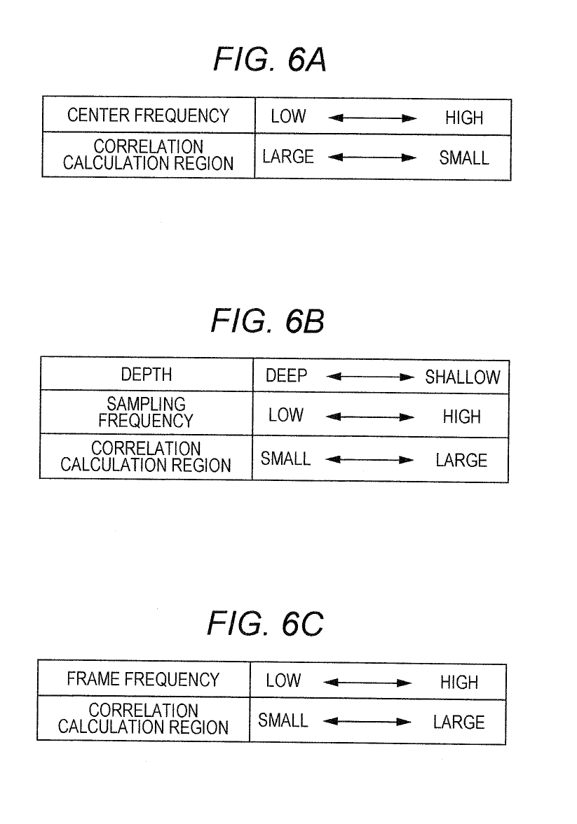

FIGS. 6A to 6C are diagrams illustrating methods of setting the size of the correlation calculation region R_{C} according to the transmission/reception setting. FIGS. 6A, 6B and 6C illustrate methods of setting the size of the correlation calculation region R_{C} according to the center frequency, the depth, and the frame frequency, respectively. Hereinafter, the setting methods will be described in order.

(Setting of Size of Correlation Calculation Region R_{C} According to Center Frequency)

First, the setting of the size of the correlation calculation region R_{C} according to the setting of the center frequency of the transmission ultrasonic wave will be described.

As expressed in the above-described Mathematical Formula (4), the phase difference between the extension-period waveform r(t) and the compression-period waveform s(t) is proportional to the center frequency, and the slope of the linear function indicating the phase difference is decreased as the center frequency is lowered.

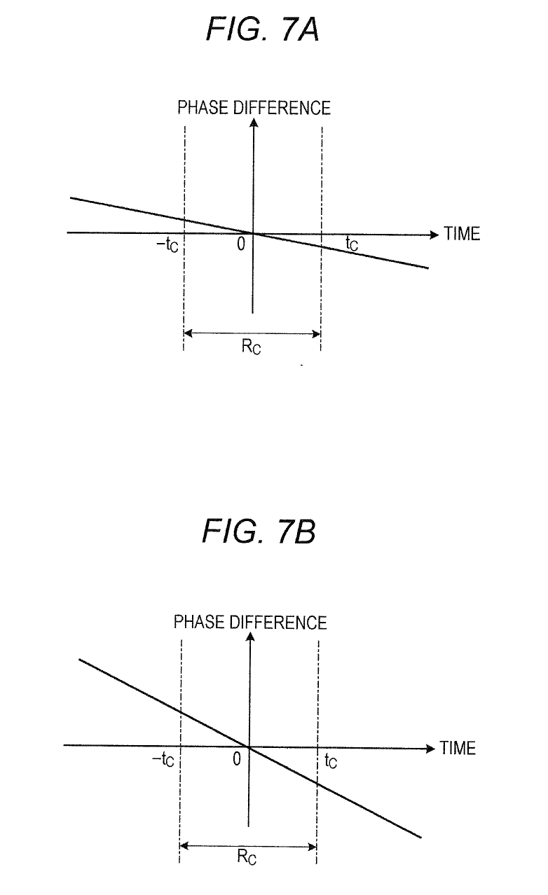

FIGS. 7A and 7B are diagrams illustrating the difference in phase difference according to the center frequency in the case where the same distortion is given. FIG. 7A illustrates the phase difference in the case where the center frequency is ω₁, and FIG. 7B illustrates the phase difference in the case where the center frequency is ω₂ higher than ω₁. In FIGS. 7A and 7B, the time range corresponding to the same correlation calculation region R_{C} is illustrated in a range from −t_{c} to t_{c}.

In any case of FIGS. 7A and 7B, noise contained in the reception signal at each time is equivalent. Therefore, if the correlation calculation region R_{C} is equal in size, in the case of FIG. 7A where the slope of the linear function indicating the phase difference is small, an S/N ratio according to a calculation result of the slope (that is, distortion) of the linear equation becomes relatively small, so that it is difficult to calculate accurate distortion.

Therefore, in the embodiment, as illustrated in FIG. 6A, as the center frequency of the transmission ultrasonic wave is lowered, a larger correlation calculation region R_{C} is set.

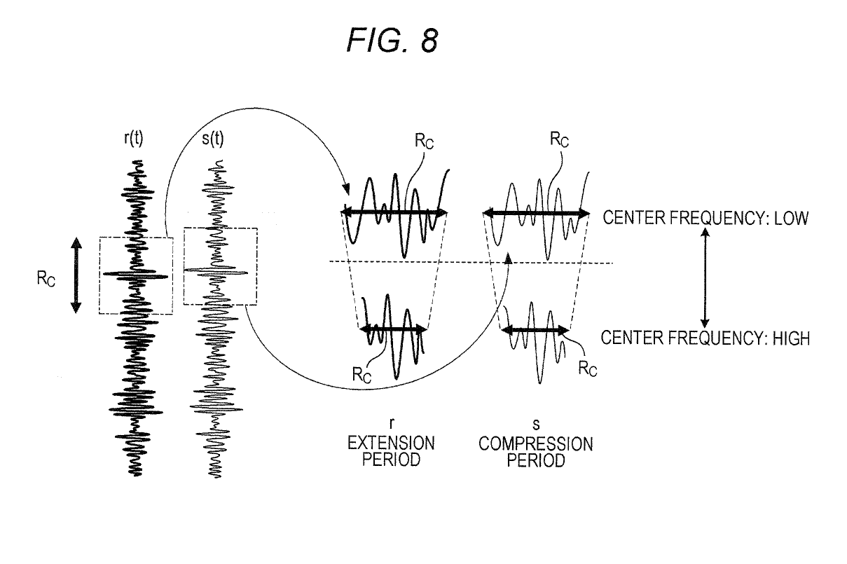

FIG. 8 is a diagram illustrating an example of setting the correlation calculation region R_{C} according to the center frequency. As illustrated on the right side of FIG. 8, in the case where the center frequency is relatively low, a larger correlation calculation region R_{C} is set. In other words, as the center frequency is lowered, a larger time range of the data is used for calculation of the distortion of one of the extension-period waveform r(t) and the compression-period waveform s(t).

Therefore, even in the case where the center frequency is low and the slope of the linear function is small, a sufficient S/N ratio according to the calculation result of the slope is secured, so that it is possible to appropriately calculate the distortion.

In addition, as the center frequency of the transmission ultrasonic wave is heightened, the correlation calculation region R_{C} is set to be smaller, so that, in the case where a sufficient S/N ratio is obtained, it is possible to improve the resolution of the elastic image representing the distribution of distortion. In addition, it is possible to shorten the time required for calculating the distortion.

The relationship between the center frequency and the size of the correlation calculation region R_{C} can be set to be typically a proportional relationship. However, for example, an increase/decrease rate of the correlation calculation region R_{C} is changed according to the center frequency, or the size of the correlation calculation region R_{C} may be set to be constant with respect to the center frequency in a predetermined range so that the S/N ratio does not fall below a predetermined value or so that the resolution falls within a predetermined range.

(Setting of Size of Correlation Calculation Region R_{C} According to Depth)

Next, the setting of the size of the correlation calculation region R_{C} according to the setting of the depth of inspection of the subject will be described.

As described above, in the ultrasonic diagnostic apparatus U according to the embodiment, the number of times of sampling in the reception process for the reflected wave is set to be constant. As the set value of the depth in the transmission/reception setting becomes larger, the sampling frequency is lowered, and as the set value of the depth becomes smaller, the sampling frequency is heightened. Herein, if the sizes of the correlation calculation regions R_{C} (that is, the number of sample data used for calculation of one distortion) is set to be equal between the two cases where the sampling frequencies are different, the sizes (lengths) of the ranges corresponding to the correlation calculation regions R_{C} in the subject are different from each other. In other words, the number of distortions calculated in the same portion of the subject (that is, the resolution of the part indicating the associated portion in the elastic image) varies with the sampling frequency. As a result, since the display image quality of the elastic image with respect to the same portion of the subject varies with the sampling frequency (set value of the depth), the display is undesirable for the user who desires to change only the depth.

Therefore, in the embodiment, as the set value of the depth is lowered (that is, as the sampling frequency is heightened), the correlation calculation region R_{C} is set to be larger. Specifically, the size of the correlation calculation region R_{C} is set to be inversely proportional to the depth and proportional to the sampling frequency. Accordingly, it possible to equalize the resolutions of the elastic images relating to the same portion of the subject irrespective of the set value of the depth.

(Setting of Size of Correlation Calculation Region R_{C} According to Frame Frequency)

Next, the setting of the size of the correlation calculation region R_{C} according to the setting of the frame frequency relating to the ultrasonic scanning will be described.

As the frame frequency becomes relatively high, the time interval between two frames used for calculating the distortion of the subject becomes small, and the distortion of the subject between the two frames becomes small. In other words, the slope of the linear function relating to the phase difference expressed in the above-described Mathematical Formula (4) and FIGS. 7A and 7B becomes small. For this reason, the S/N ratio according to the calculation result of the slope (that is, distortion) of the linear function becomes relatively small, so that it is difficult to calculate accurate distortion.

Therefore, in the embodiment, as the frame frequency is heightened, the correlation calculation region R_{C} is set to be larger. Accordingly, even in the case where the frame frequency is high and the slope of the linear function is small, a sufficient S/N ratio according to the calculation result of the slope is secured, so that it is possible to appropriately calculate the distortion.

In addition, as the frame frequency is lowered, the correlation calculation region R_{C} is set to be smaller, so that, in the case where a sufficient S/N ratio is obtained, it possible to improve the resolution of the elastic image representing the distribution of distortion. In addition, it is possible to shorten the time required for calculating the distortion.

The relationship between the frame frequency and the size of the correlation calculation region R_{C} can be set to be a typically proportional relationship. However, for example, an increase/decrease rate of the correlation calculation region R_{C} is changed according to the frame frequency or the size of the correlation calculation region R_{C} may be set to be constant with respect to the frame frequency in a predetermined range so that the S/N ratio does not fall below a predetermined value or so that the resolution falls within a predetermined range.

In the above description, the method of setting the size of the correlation calculation region R_{C} for each of the center frequency, the depth, and the frame frequency has been described. However, in the ultrasonic diagnostic apparatus U according to the embodiment, for all available combinations of the center frequency, the depth, and the frame frequency, the sizes of the optimal correlation calculation regions R_{C} are calculated in advance and stored as the calculation parameter table 15a in the HDD of the control unit 15.

Subsequently, the elastic image display process and the elastic image generation process performed by the ultrasonic diagnostic apparatus U will be described.

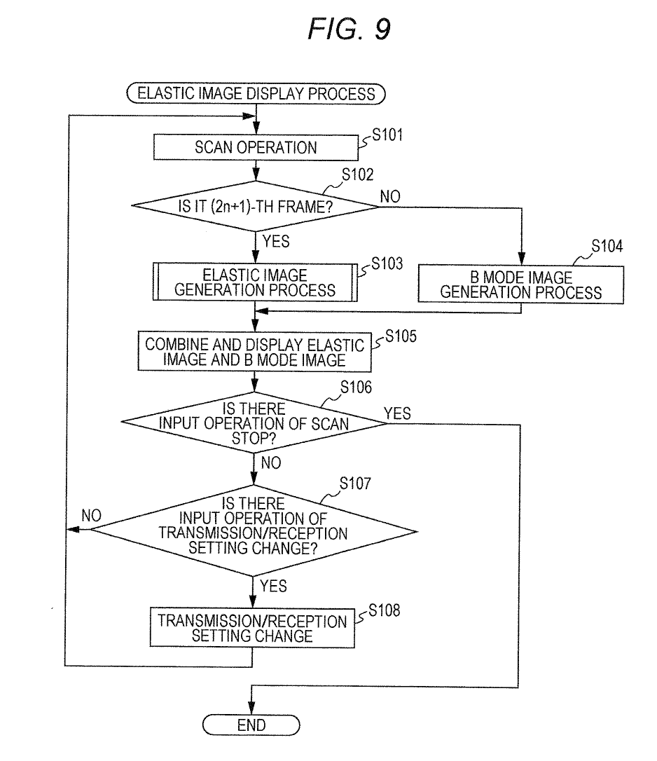

FIG. 9 is a flowchart illustrating a control procedure of the control unit 15 in the elastic image display process.

The elastic image display process is performed in the case where the elasticity information display mode is selected by a user's input operation to the operation input unit 18 or the like in a measurement display process relating to the ultrasonic diagnosis.

When the elastic image display process is started, the control unit 15 performs a scan operation, that is, a transmission/reception operation relating to one-time scanning of the ultrasonic wave on the basis of the transmission/reception setting (step S101: transmission/reception step). Herein, the control unit 15 allows the transmission unit 12 to output a pulse signal to the ultrasonic probe 2, so that the ultrasonic probe 2 performs ultrasonic scanning and transmission at the center frequency and the frame frequency determined by the transmission/reception setting. The control unit 15 allows the reception unit 13 to acquire the reception signal relating to the reflected wave received by the ultrasonic probe 2 at the sampling frequency corresponding to the depth determined by the transmission/reception setting and stores the obtained frame data in the storage unit 17.

The control unit 15 determines whether or not the frame relating to the most recent scanning operation is the (2n+1)-th frame from the scan start (step S102). In the case where it is determined that the frame is the (2n+1)-th frame (“YES” in step S102), the control unit 15 performs an elastic image generation process described later and stores the elastic image in the storage unit 17 (step S103: elasticity information generating step). In addition, in the case where it is determined that the frame is not the (2n+1)-th frame (that is, the frame is the 2n-th frame or the first frame) (“NO” in step S102), the control unit 15 allows the image processing unit 16 to generate the image data of the B mode image on the basis of the frame data and stores the image data in the storage unit 17 (step S104). Furthermore, with respect to the first frame, only the acquisition of the frame data is performed, and thus, the generation of the B mode image may not be performed.

The control unit 15 allows the image processing unit 16 to generate combined image data of the most recent elastic image and the B mode image and allows the output display unit 19 to display the image relating to the combined image data (step S105: display step).

The control unit 15 determines whether or not an input operation instructing to stop the scanning operation is performed on the operation input unit 18 (step S106). In the case where it is determined that the input operation is performed (“YES” in step S106), the control unit 15 ends the elastic image display process.

In the case where it is determined that the input operation instructing the stop of the scanning operation is not performed (“NO” in step S106), the control unit 15 allows the operation input unit 18 to input an input operation instructing to change the transmission/reception setting (step S107).

In the case where it is determined that the input operation is not performed (“NO” instep S107), the control unit 15 allows the process to proceed to step S101.

In the case where it is determined that the input operation instructing the change of the transmission/reception setting is performed (“YES” at step S107), the control unit 15 changes and updates the transmission/reception setting according to the input operation (step S108). If the process of step S108 is ended, the control unit 15 allows the process to proceed to step S101.

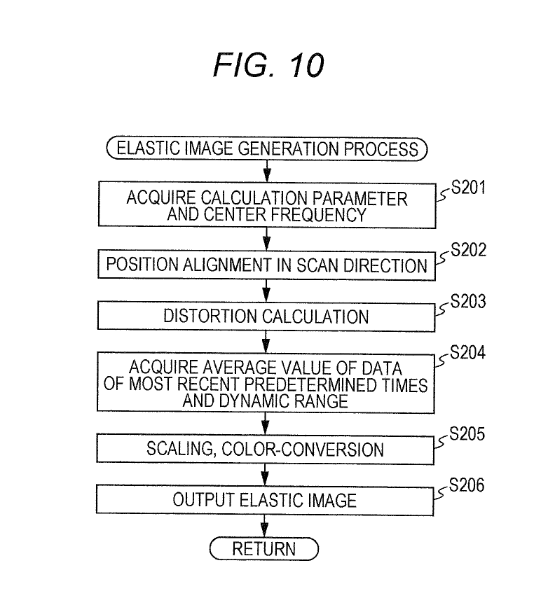

FIG. 10 is a flowchart illustrating a control procedure of the processing control unit 16a in the elastic image generation process.

When the elastic image generation process is started, the processing control unit 16a (CPU) of the image processing unit 16 calculates the calculation parameter (the size of the correlation calculation region R_{C}) and the value of the center frequency of the transmission ultrasonic wave relating to the current transmission/reception setting by referring to the calculation parameter table 15a (step S201).

The processing control unit 16a acquires frame data relating to two adjacent odd-numbered frames and performs positioning in the scan direction of the ultrasonic probe 2 (step S202). The processing control unit 16a determines a combination of the scanning positions corresponding to the same position by adjustment to positions which are set in advance, pattern matching, or the like.

The processing control unit 16a calculates the distortion according to a change in the pressurized state for each correlation calculation region R_{C} on the basis of the above-described algorithm by using a combination of the ultrasonic waveform (extension-period waveform) acquired in the case where a small force is applied to the subject at each scanning position and the ultrasonic waveforms (compression-period waveforms) acquired in the case where a large force is applied to the subject and the value of the center frequency of the transmission ultrasonic wave (step S203). In the calculation of the distortion, the processing control unit 16a inverts the sign of the distortion value in the case where the first reception waveform data among the reception waveform data relating to the two frame data are a compression-period waveform. In the process of step S203, after the distortion is calculated in all the correlation calculation regions R_{C} for one frame to generate two-dimensional data relating to the distortion, smoothing of the distortion data in a two-dimensional in-plane direction may be performed on the basis of predetermined setting. Smoothing in a time axis direction may also be performed.

The processing control unit 16a acquires an average value and a dynamic range of the distortion data acquired the most recent predetermined number of times (step S204). These values can be easily acquired by storing the values in RAMs or the like of a predetermined number of the processing control units 16a every time final elastic image data are output in each elastic image generation process.

The processing control unit 16a scales the value of the distortion on the basis of the acquired value acquired this time, the average value of the most recent predetermined number of times, and the dynamic range. In addition, the processing control unit 16a converts the distortion data into color data corresponding to the magnitude of the distortion according to predetermined display setting (step S205). For example, the largest distortion in the dynamic range is set to red, the smallest distortion is set to blue, and intermediate distortion is set to an intermediate color between red and blue according to the magnitude of the distortion.

The processing control unit 16a outputs the data of a two-dimensional image (elastic image) relating to the scaled, color-converted distortion and stores the data in the storage unit 17 (step S206).

If the process of step S206 is ended, the processing control unit 16a ends the elastic image generation process.

As described above, the ultrasonic diagnostic apparatus U according to the embodiment is configured to include the ultrasonic probe 2 transmitting the ultrasonic wave to the subject and receiving the reflected waves of the transmitted ultrasonic wave, the transmission unit 12, the reception unit 13, and the control unit 15 (transmission/reception control unit) allowing the ultrasonic probe 2 to transmit the ultrasonic wave and acquiring the reception signal relating to the ultrasonic wave received by the ultrasonic probe 2 on the basis of the predetermined transmission/reception setting, and the processing control unit 16a (calculation parameter acquisition unit) acquiring the calculation parameter which is used for generating the elasticity information in the subject on the basis of the reception signal and is determined in correspondence with the transmission/reception setting. The processing control unit 16a generates the elasticity information by using the reception signal on the basis of the acquired calculation parameter (elasticity information generation unit). The control unit 15 allows the output display unit 19 to display the elasticity information (display control unit). According to the configuration, by using an appropriate calculation parameter corresponding to the transmission/reception setting, even in the case where the transmission/reception setting is changed, it is possible to generate and display the elasticity information with an appropriate, stable quality. In addition, since the calculation parameter used for generating the elasticity information is to be relating to the transmission/reception setting, it is possible to easily acquire an appropriate calculation parameter on the basis of the transmission/reception setting. Therefore, according to the ultrasonic diagnostic apparatus U according to the embodiment, it is possible to easily generate appropriate elasticity information in an arbitrary transmission/reception setting.

The ultrasonic diagnostic apparatus U is configured to further include the control unit 15 storing, in the HDD as a calculation parameter table 15a, the sizes of a plurality of the correlation calculation regions R_{C} determined in advance in association with a plurality of different transmission/reception settings. The processing control unit 16a acquires the sizes of the correlation calculation regions RC from the calculation parameter table 15a of the control unit 15 (operation parameter acquisition unit). Therefore, it is possible to easily acquire the sizes of the appropriate correlation calculation regions R_{C} by referring to the calculation parameter table 15a. In addition, it is possible to simplify the process in the generation of the elasticity information by obtaining the sizes of the correlation calculation regions R_{C} in advance and storing the sizes in the control unit 15.

In addition, the processing control unit 16a calculates the distortion of the subject to generate the elasticity information for each of the correlation calculation regions R_{C} corresponding to the predetermined time range among the extension-period waveform r(t) and compression-period waveform s(t) by using the extension-period waveform r(t) (first reception signal) relating to the ultrasonic wave reflected by the subject before compression (first pressurized state) and the compression-period waveform s(t) (second reception signal) relating to the ultrasonic wave reflected by the subject after the compression (second pressurized state) (elasticity information generation unit). The processing control unit 16a acquires the calculation parameter representing the sizes of the correlation calculation regions R_{C} (calculation parameter acquisition unit). According to the configuration, since the correlation calculation regions R_{C} having appropriate sizes are set corresponding to the transmission/reception setting and the distortion of the subject is calculated for each correlation calculation region R_{C}, it is possible to calculate appropriate distortion with appropriate accuracy and resolution in arbitrary transmission/reception setting.

In addition, the processing control unit 16a extracts the phase difference component at each time in the time range corresponding to the correlation calculation region R_{C} between the extension-period waveform r(t) and the compression-period waveform s(t) and calculates the distortion of the subject in the correlation calculation region R_{C} from the phase difference component and the center frequency of the ultrasonic wave transmitted from the ultrasonic probe 2 (elasticity information generation unit). Accordingly, it is possible to accurately calculate the distortion of the subject from the extension-period waveform r(t) and the compression-period waveform s(t) by an easy process.

In addition, the transmission/reception setting includes the setting of the center frequency of the ultrasonic wave transmitted from the ultrasonic probe 2. Accordingly, since the correlation calculation region R_{C} having a magnitude corresponding to the center frequency is set, a sufficient S/N ratio is secured, so that it is possible to appropriately calculate the distortion. In addition, in the case where a sufficient S/N ratio is obtained, it is possible to improve the resolution of the elastic image representing the distribution of distortion. In addition, it is possible to shorten the time required for calculating the distortion.

In addition, the reception unit 13 and the control unit 15 acquire a reception signal relating to a reflected wave of which reflection position on the subject is equal to or less than a predetermined maximum depth among the ultrasonic waves transmitted to the subject (transmission/reception control unit). The transmission/reception setting includes the setting of the maximum depth. In addition, the reception unit 13 and the control unit 15 acquire the reception signal at a sampling frequency that is lower as the set maximum depth is larger (transmission/reception control unit). Accordingly, since the correlation calculation region R_{C} having a size corresponding to the set depth is set, it is possible to equalize the resolutions of the elastic images relating to the same portion of the subject irrespective of the set value of the depth.

In addition, the transmission unit 12, the reception unit 13, and the control unit 15 allows the ultrasonic probe 2 to transmit the ultrasonic wave, while scanning the ultrasonic wave in a predetermined scan direction, and acquires the two-dimensional data of the reception signal relating to the scanned ultrasonic wave every scanning (transmission/reception control unit). The transmission/reception setting includes the setting of the frame frequency indicating a frequency of the scanning. Accordingly, since the correlation calculation region R_{C} having a size corresponding to the frame frequency is set, a sufficient S/N ratio is secured for the magnitude of the distortion according to the frame frequency, so that it is possible to appropriately calculate the distortion. In addition, in the case where a sufficient S/N ratio is obtained, it is possible to improve the resolution of the elastic image representing the distribution of distortion. In addition, it is possible to shorten the time required for calculating the distortion.

In addition, the ultrasonic diagnostic apparatus U is configured to include an operation input unit 18 that accepts an input operation for determining transmission/reception setting. The control unit 15 determines the transmission/reception setting on the basis of the input operation (transmission/reception setting change unit). According to the configuration, it is possible to change the transmission/reception setting by the input operation to the operation input unit 18. In addition, since the correlation calculation region R_{C} having an appropriate size is set according to the change of the transmission/reception setting and the distortion is calculated, it is possible for the user to easily obtain appropriate elasticity information without performing a special operation.

In addition, the elasticity information is an elastic image representing a distribution of values relating to distortion in the subject. Accordingly, it is possible to display the elasticity information that is visually easy to understand.

In addition, the processing control unit 16a generates the B mode image representing the internal structure of the subject by using the reception signal (ultrasonic image generation unit). The control unit 15 allows the output display unit 19 to display the B mode image and the elastic image (display control unit). Accordingly, it is possible to display the internal structure of the subject and the information on the hardness so as to be easy to understand visually.

In addition, the control unit 15 allows the output display unit 19 to display the B mode image and the elastic image in a superimposed manner (display control unit). Accordingly, it is possible to display the internal structure of the subject and the information on the hardness so as to be easy to understand visually and to be easy to compare. In addition, in the case where the resolution of the elastic image is lower than that of the B mode image, it is possible to display a tendency of the distortion distribution in the internal structure by the elastic image while displaying the internal structure of the subject at a high resolution by the B mode image.

A method of controlling an ultrasonic diagnostic apparatus U according to the embodiment is configured to include a transmission/reception step of allowing an ultrasonic probe 2 to transmit an ultrasonic wave and receiving a reception signal relating to the ultrasonic wave received by the ultrasonic probe 2 on the basis of predetermined transmission/reception setting, a calculation parameter acquisition step of acquiring a calculation parameter which is used for generation of elasticity information in a subject based on the reception signal and is determined in correspondence with the transmission/reception setting; an elasticity information generation step of generating elasticity information by using the reception signal on the basis of the acquired calculation parameter, and a display step of allowing an output display unit 19 to display the elasticity information. Accordingly, it is possible to easily generate appropriate elasticity information in arbitrary transmission/reception setting.

A program according to the embodiment causes an ultrasonic diagnostic apparatus U (computer) to function as a transmission/reception control unit of allowing an ultrasonic probe 2 to transmit an ultrasonic wave to a subject and receiving a reception signal relating to the ultrasonic wave received by the ultrasonic probe 2 on the basis of predetermined transmission/reception setting, a calculation parameter acquisition unit of acquiring a calculation parameter which is used for generation of elasticity information in the subject based on the reception signal and is determined in correspondence with the transmission/reception setting; an elasticity information generation unit of generating elasticity information by using the reception signal on the basis of the acquired calculation parameter, and a display control unit of allowing an output display unit 19 to display the elasticity information. Accordingly, it is possible to easily generate appropriate elasticity information in arbitrary transmission/reception setting.

### Second Embodiment

Subsequently, a second embodiment of the present invention will be described.

The embodiment is different from the first embodiment in terms of a distortion calculation algorithm. Hereinafter, the differences from the first embodiment will be described.

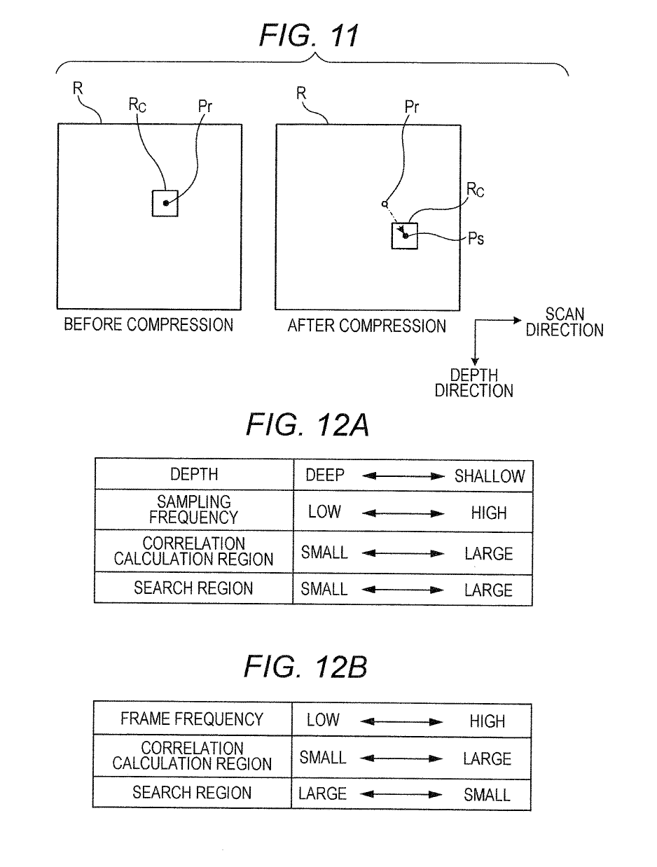

FIG. 11 is a diagram for explaining a distortion calculation method according to the embodiment.

The left side of FIG. 11 illustrates a predetermined portion (search region R) of the data (first two-dimensional data) relating to one frame data out of two frame data used for calculating the distortion in the case where the force applied to the subject is small. The right side of FIG. 11 illustrates the same range (search region R) in the other frame data (second two-dimensional data). Hereinafter, the left side of FIG. 11 is also referred to as the search region R relating to the subject before compression, and the right side is also referred to as the search region R relating to the subject after compression. In addition, the left/right direction in FIG. 11 indicates the scan direction of the ultrasonic wave, and the up/down direction indicates the depth direction (transmission direction of the ultrasonic wave) in the inspection of the subject.

As illustrated in FIG. 11, in the embodiment, the correlation calculation region R_{C} is set as a two-dimensional data region in the scan direction and the depth direction. In addition, the search region R is set in a predetermined range including the correlation calculation region R_{C} around the correlation calculation region R_{C} in the before-compression data as a center.

In the calculation of the distortion in the embodiment, for each correlation calculation region R_{C} in the before-compression data, it is specified which position in the after-compression search region R the correlation calculation region R_{C} is shifted to. Accordingly, the shift between the position of the representative point Pr in the before-compression correlation calculation region R_{C} and the position of the representative point Ps in the after-compression correlation calculation region R_{C} is calculated. When the shift is obtained at each point of the frame data, the distortion is calculated by spatially differentiating the shift.

FIGS. 12A and 12B are diagrams illustrating methods of setting the size of the correlation calculation region R_{C} according to the transmission/reception setting in the embodiment. FIGS. 12A and 12B illustrate methods of setting the size of the correlation calculation region R_{C} according to the depth and the frame frequency, respectively.

As illustrated in FIG. 12A, in the embodiment, the correlation calculation region R_{C} and the search region R are set to be large as the set value of the depth is smaller (that is, as the sampling frequency is higher). Specifically, the size of the correlation calculation region R_{C} and the size of the search region R are set to be inversely proportional to the depth and proportional to the sampling frequency. Accordingly, it is possible to equalize the resolutions of the elastic images relating to the same portion of the subject irrespective of the set value of the depth.

In addition, as illustrated in FIG. 12B, in the embodiment, the correlation calculation region R_{C} is set to be large as the frame frequency is higher. As a result, even in the case where the frame frequency is high and the shift between the frames is small, by increasing the correlation calculation region R_{C}, it is possible to specify the shift and calculate the distortion at a good accuracy. In addition, as the frame frequency is lower, the search region R is set to be large, and thus, in the case where the frame frequency is low and the shift between the frames is large, it is possible to suppress the occurrence of the problem in that the position of the after-compression correlation calculation region R_{C} in the search region R cannot be specified. In FIGS. 12A and 12B, one of the correlation calculation region R_{C} and the search region R may be fixed and the other may be adjusted.

As described above, in the ultrasonic diagnostic apparatus U according to the embodiment, the control unit 15 allows the ultrasonic probe 2 to transmit an ultrasonic wave while scanning the ultrasonic wave in a predetermined scan direction and acquires two-dimensional data of a reception signal relating to the scanned ultrasonic wave every scanning (transmission/reception control unit). The processing control unit 16a detects detect a shift at each position of the subject between before compression and after compression by using first two-dimensional data relating to the ultrasonic wave reflected by the subject before compression (first pressurized state) and second two-dimensional data relating to the ultrasonic wave reflected by the subject after compression (second pressurized state) (elasticity information generation unit). The shift is detected by specifying the shift in a predetermined search region R including correlation calculation regions R_{C} for each two-dimensional predetermined correlation calculation region R_{C} in the two-dimensional data. The processing control unit 16a acquires calculation parameters indicating at least one of the size of the correlation calculation region R_{C} and the size of the search region (calculation parameter acquisition unit). According to the configuration, since the correlation calculation region R_{C} having an appropriate size and the search region R having an appropriate size are set corresponding to transmission/reception setting and the distortion of the subject is calculated for each correlation calculation region R_{C}, it is possible to calculate the distortion with appropriate accuracy and resolution in arbitrary transmission/reception setting.

The present invention is not limited to the above-described embodiments, but various modifications are available.

For example, in each of the above-described embodiments, the case of obtaining the distortion of a living tissue by using the present invention as a medical device has been described as an example. However, the object of the distortion calculation is not limited to the living tissue. The present invention can be appropriately used for building structures or various kinds of products having a compact structure where a pressure is properly applied to an inner object.

In each of the above-described embodiments, the ultrasonic diagnostic apparatus U that calculates the distortion from the reception signals of the reflected waves before and after compression by changing the pressure with which the ultrasonic probe 2 is pressed has been described as an example. However, the present invention is not intended to be limited thereto. For example, the present invention may be applied to an ultrasonic diagnostic apparatus using a technique (ARFI: acoustic radiation force impulse) that transmits a strong sound wave (shear wave) for pressurization in parallel with an ultrasonic wave for inspection transmitted from an ultrasonic probe and acquires a distribution of elastic modulus of a subject on the basis of a difference in propagation speed of the sound wave according to hardness of the subject.

In each of the above embodiments, the case where the calculation parameter table 15a is stored in the HDD of the control unit 15 and the calculation parameter is acquired with reference to the calculation parameter table 15a has been described as an example. However, alternatively, the control unit 15 (or the processing control unit 16a) may calculate the calculation parameter on the basis of the transmission/reception setting and predetermined calculation formulas.

In addition, the calculation parameter table 15a may be stored in a storage device outside the ultrasonic diagnostic apparatus U, and the calculation parameter maybe acquired from the storage device through a communication unit (not shown).

In each of the above-described embodiments, the elastic image representing the two-dimensional distribution of distortion has been described as an example. However, the elastic image may be another image relating to distortion of the subject according to pressurization on the subject. For example, the elastic image may be an image representing a distribution of elastic modulus or a distribution of shift of each component. Namely, the value relating to the distortion represented by the elastic image may be elastic modulus or shift other than the distortion.

In each of the above-described embodiments, the case where the elastic image as the elasticity information is displayed on the output display unit 19 has been described as an example. However, the elasticity information is not limited to such an elasticity image. For example, in an aspect, the elasticity information may be statistical information such as a histogram, a standard deviation, and a representative value (average value or median value) of a distribution of distortion, and such information may displayed on the output display unit 19 as a graph or a text.

In each of the above embodiments, the center frequency, the depth, and the frame frequency has been exemplified as the transmission/reception setting. However, calculation parameters may be set in association with other setting relating to the transmission and reception of the ultrasonic wave.

In each of the above-described embodiments, the case where the elastic image is generated on the basis of reception signals of odd-numbered frames and the B mode image is generated on the basis of reception signals of even-numbered frames has been described as an example. However, alternatively, the reception signal of each frame may be used both for generating the elastic image and for generating the B mode image.

In addition, the output destination of the elastic image is not limited to the display screen of the output display unit 19, but an external device or an external display may be used. In addition, directly output to printing may be used, or instead of image data, numerical data may be output to an external device.

In addition, the image processing unit 16 according to the embodiment may be provided independently of the ultrasonic probe 2 and other portions of the ultrasonic diagnostic apparatus main body 1. Namely, the image processing unit may be a dedicated signal processing device. In addition, signal processing of the present invention can be realized by typical software processing. Software may be installed in a computer such as a general PC, and a control unit (CPU) of the computer may execute the software by using input waveform data.

Although the present invention has been described and illustrated in detail, it is clearly understood that the same is by way of illustrated and example only and is not to be taken by way of limitation, the scope of the present invention being interpreted by terms of the appended claims.

What is claimed is:

1. An ultrasonic diagnostic apparatus comprising:
an ultrasonic probe which transmits an ultrasonic wave to a subject and receives a reflected wave of the transmitted ultrasonic wave;
a transmission circuit which allows the ultrasonic probe to transmit the ultrasonic wave and a reception circuit which acquires a reception signal relating to the ultrasonic wave received by the ultrasonic probe on the basis of predetermined transmission/reception setting; and
a processor;
wherein the processor acquires a calculation parameter which is used for generation of elasticity information in the subject based on the reception signal and is determined in correspondence with the transmission/reception setting;
wherein the processor generates the elasticity information by using the reception signal on the basis of the acquired calculation parameter; and
wherein the processor allows a display to display the elasticity information.

2. The ultrasonic diagnostic apparatus according to claim 1, further comprising
a memory which stores a plurality of the calculation parameters determined in advance in association with a plurality of different transmission/reception settings,
wherein the processor acquires the calculation parameter from the memory.

3. The ultrasonic diagnostic apparatus according to claim 1,
wherein the processor generates the elasticity information by calculating distortion of the subject for each correlation calculation region corresponding to a predetermined time range between a first reception signal relating to the ultrasonic wave reflected by the subject in a first pressurized state and a second reception signal relating to the ultrasonic wave reflected by the subject in a second pressurized state by using the first reception signal and the second reception signal, and
the processor acquires the calculation parameter indicating a size of the correlation calculation region.

4. The ultrasonic diagnostic apparatus according to claim 3, wherein the processor extracts a phase difference component at each time in a time range corresponding to the correlation calculation region between the first reception signal and the second reception signal and calculates the distortion of the subject in the correlation calculation region from the phase difference component and a center frequency of the ultrasonic wave transmitted from the ultrasonic probe.

5. The ultrasonic diagnostic apparatus according to claim 1,
wherein the transmission circuit allows the ultrasonic probe to transmit an ultrasonic wave while scanning the ultrasonic wave in a predetermined scan direction and the reception circuit acquires two-dimensional data of the reception signal relating to the scanned ultrasonic wave every scanning,
the processor generates the elasticity information by detecting a shift at each position of the subject between a first pressurized state and a second pressurized state by using first two-dimensional data relating to the ultrasonic wave reflected by the subject in the first pressurized state and second two-dimensional data relating to the ultrasonic wave reflected by the subject in the second pressurized state,
the detection of the shift is performed by specifying the shift in a predetermined search region including the correlation calculation region for each of two-dimensional predetermined correlation calculation regions in the two-dimensional data, and
the processor acquires the calculation parameter indicating at least one of a size of the correlation calculation region and a size of the search region.

6. The ultrasonic diagnostic apparatus according to claim 1, wherein the transmission/reception setting includes setting of a center frequency of an ultrasonic wave transmitted from the ultrasonic probe.

7. The ultrasonic diagnostic apparatus according to claim 1,
wherein the reception circuit acquires the reception signal relating to the reflected wave of which reflection position on the subject is equal to or less than a predetermined maximum depth among the ultrasonic waves transmitted to the subject, and
the transmission/reception setting includes setting of the maximum depth.

8. The ultrasonic diagnostic apparatus according to claim 7, wherein the reception circuit acquires the reception signal at a sampling frequency that is lower as the set maximum depth is larger.

9. The ultrasonic diagnostic apparatus according to claim 1,
wherein the transmission circuit allows the ultrasonic probe to transmit while scanning the ultrasonic wave in a predetermined scan direction and the reception circuit acquires two-dimensional data of the reception signal relating to the scanned ultrasonic wave every scanning, and
the transmission/reception setting includes setting of a frame frequency indicating a frequency of the scanning.

10. The ultrasonic diagnostic apparatus according to claim 1, further comprising:
an input device which receives an input operation for determining the transmission/reception setting; and
the processor determines the transmission/reception setting on the basis of the input operation.

11. The ultrasonic diagnostic apparatus according to claim 1, wherein the elasticity information is an elastic image representing a distribution of values relating to distortion in the subject.

12. The ultrasonic diagnostic apparatus according to claim 11, wherein the processor generates an ultrasonic image representing an internal structure of the subject by using the reception signal,
wherein the processor allows the display to display the ultrasonic image and the elastic image.

13. The ultrasonic diagnostic apparatus according to claim 12, wherein the processor allows the display to display the ultrasonic image and the elastic image in a superimposed manner.

14. A method of controlling an ultrasonic diagnostic apparatus including an ultrasonic probe which transmits an ultrasonic wave to a subject and receives a reflected wave of the transmitted ultrasonic wave, the method comprising:
allowing the ultrasonic probe to transmit an ultrasonic wave and acquiring a reception signal relating to the ultrasonic wave received by the ultrasonic probe on the basis of predetermined transmission/reception setting;
acquiring a calculation parameter which is used for generation of elasticity information in the subject based on the reception signal and is determined in correspondence with the transmission/reception setting;
generating the elasticity information by using the reception signal on the basis of the acquired calculation parameter; and
allowing a display to display the elasticity information.

15. A non-transitory recording medium storing a computer readable program, the program causing a computer to function as:
a transmission circuit which allows an ultrasonic probe to transmit an ultrasonic wave to a subject and a reception circuit which acquires a reception signal relating to a reflected wave of the ultrasonic wave received by the ultrasonic probe on the basis of predetermined transmission/reception setting;
a processor acquires a calculation parameter which is used for generation of elasticity information in the subject based on the reception signal and is determined in correspondence with the transmission/reception setting;
wherein the processor generates the elasticity information by using the reception signal on the basis of the acquired calculation parameter; and
wherein the processor allows a display to display the elasticity information.
